(19) United States
(12) Patent Application Publication
(10) Pub. No.: US 2008/0015439 A1
(43) Pub. Date: Jan. 17, 2008

(54) Method and Apparatus for Measuring and/or Detecting Flow Behavior of a Body Fluid Using Ultrasound

(75) Inventors: Balasundara Raju, Tarrytown, NY (US); Eric Cohen-Solal, Ossining, NY (US); Shervin Ayati, Carlisle, MA (US)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics, N. V., Eindhoven (NL)

(21) Appl. No.: 11/574,885

(22) PCT Filed: Sep. 8, 2005
(86) PCT No.: PCT/IB05/52938

§ 371 Date: Mar. 8, 2007

Related U.S. Application Data
(60) Provisional application No. 60/609,676, filed on Sep. 13, 2004.

(51) Int. Cl. A61B 8/14

(52) U.S. Cl. 600/455

(57) ABSTRACT

An ultrasound method and apparatus for detecting and/or measuring the pulse and/or blood flow of a subject calculates a Doppler signal spectrum from an ultrasound signal backscattered from the blood in an artery of the subject. Indicia of flow behavior are calculated for several frequency slices within the Doppler signal spectrum and these indicia may be used to determine pulsatility and/or blood flow, as well as other parameters of flow behavior. Because of the robust nature of the calculated indicia, the ultrasound method and apparatus has particular use in an Automated or Semi-Automated External Defibrillator (AED) for determining whether to defibrillate a patient.

The present invention relates generally to the field of medical ultrasound diagnostics and, more specifically, to a method and apparatus for measuring and/or detecting the flow behavior of a body fluid using an externally attached ultrasound device.

In emergencies and during operative procedures, the assessment of the pulse state of the patient is essential for both diagnosis of the problem and determining the appropriate therapy for the problem. The presence of a cardiac pulse in a patient is typically detected by palpating the patient's neck and sensing palpable pressure changes due to the change in the patient's carotid artery volume. When the heart's ventricles contract during a heartbeat, a pressure wave is sent throughout the patient's peripheral circulation system. A carotid pulse waveform rises with the ventricular ejection of blood at systole and peaks when the pressure wave from the heart reaches a maximum. The carotid pulse falls off again as the pressure subsides toward the end of the pulse.

The absence of a detectable cardiac pulse in a patient is a strong indicator of cardiac arrest. Cardiac arrest is a life-threatening medical condition in which the patient's heart fails to provide blood flow to support life. During cardiac arrest, the electrical activity of the heart may be disorganized (ventricular fibrillation), too rapid (ventricular tachycardia), absent (asystole), or organized at a normal or slow heart rate without producing blood flow (pulseless electrical activity).

The form of therapy to be provided to a patient in cardiac arrest depends, in part, on an assessment of the patient's cardiac condition. For example, a caregiver may apply a defibrillation shock to a patient experiencing ventricular fibrillation (VF) or ventricular tachycardia (VT) to stop the unsynchronized or rapid electrical activity and allow a perfusing rhythm to return. External defibrillation, in particular, is provided by applying a strong electric shock to the patient's heart through electrodes placed on the surface of the patient's body. If the patient lacks a detectable pulse and is experiencing asystole or pulseless electrical activity (PEA), defibrillation cannot be applied and the caregiver may perform cardiopulmonary resuscitation (CPR), which causes some blood to flow in the patient.

Before providing therapy such as defibrillation or CPR to a patient, a caregiver must first confirm that the patient is in cardiac arrest. In general, external defibrillation is suitable only for patients that are unconscious, apneic, pulseless, and in VF or VT. Medical guidelines indicate that the presence or absence of a cardiac pulse in a patient should be determined within 10 seconds. For example, the American Heart Association protocol for cardiopulmonary resuscitation (CPR) requires a healthcare professional to assess the patient's pulse for five to ten seconds. Lack of a pulse is an indication for the commencement of external chest compressions. Assessing the pulse, while seemingly simple on a conscious adult, is the most often failed component of a basic life support assessment sequence, which may be attributed to a variety of reasons, such as lack of experience, poor landmarks, or a bias to either finding or not finding a pulse. Failure to accurately detect the presence or absence of the pulse will lead to adverse treatment of the patient either when providing or not providing CPR or defibrillation therapy to the patient.

Electrocardiogram (ECG) signals are normally used to determine whether or not a defibrillating shock should be applied. However, certain rhythms that a rescuer is likely to encounter cannot be determined solely by the ECG signal, e.g. pulseless electrical activity; diagnoses of these rhythms require supporting evidence of a lack of perfusion despite the myocardial electrical activity as indicated by the ECG signal.

Because the pulse check or blood flow measurement is performed manually, it is subject to human error, and in an emergency situation where time is of the essence, the amount of time taken for the manual pulse state assessment is too long thereby causing detrimental results. A reliable pulse state assessment device is needed to solve these limitations.

Even when the ECG analysis is performed, it is possible that the results may mislead the rescuer into taking the wrong course of action. For instance, after cardiac arrest, the patient may enter a state of pulseless electrical activity (PEA) where the ECG will register normal electrical activity, but there is no pulse present. Because the ECG analysis shows a “pulse” (i.e., electrical activity), the rescuer would take no further action, thereby gravely endangering the patient. Conversely, if a rescuer incorrectly concludes that the patient has no pulse (because of a necessarily rushed preliminary evaluation or false determination of PEA), and proceeds to provide therapy, such as CPR, the patient's chance for recovery of circulation is curtailed.

Thus, in order for a rescuer to quickly determine whether or not to provide therapy to a patient, it is necessary to develop an integrated system that is quickly and easily able to analyze the patient's pulse, the amount of blood flow, and perhaps the ECG signals in order to correctly determine whether there is any pulsatile flow in the arteries of the patient.

This necessity is particularly dire in situations or systems in which the rescuer is untrained and/or inexperienced person, as is the case in the system described in U.S. Pat. No. 6,575,914 to Rock et al., which is assigned to the same assignee as the present invention, is hereby incorporated by reference in its entirety, and will be referred to hereafter as “the Rock patent”. The Rock patent discloses an Automated External Defibrillator (AED) (hereinafter both AEDs and Semi-Automated External Defibrillators—SAEDs—will be referred to jointly as AEDs) which can be used by first-responding caregivers with little or no medical training to determine whether or not to apply defibrillation to an unconscious patient.

The Rock AED has a defibrillator, a sensor pad for transmitting and receiving Doppler ultrasound signals, two sensor pads for obtaining an ECG signal, and a processor which receives and assesses the Doppler and ECG signals in order to determine whether defibrillation is appropriate for the patient (i.e., whether or not there is a pulse). The Doppler pad is adhesively secured to a patient's skin above the carotid artery to sense the carotid pulse (which is a key indicator of sufficient blood pulsatile flow).

Specifically, the processor in the Rock AED analyzes the Doppler signals to determine whether there is a detectable pulse and analyzes the ECG signals to determine whether there is a “shockable rhythm” (see, e.g., FIG. 7 and accompanying description at col. 6, line 60, to col. 7, line 52, in the Rock patent). Based on the results of these two separate analyses, the processor determines whether to advise defibrillation or not (id.). Although the Rock patent discusses “integrating” the Doppler and ECG signals, the processor in the Rock AED merely considers the results of both analyses and does not integrate, either mathematically or analytically, the Doppler and ECG signal analyses.

The determination of a detectable pulse by the processor in the Rock AED is made by comparing the received Doppler signals against “a threshold statistically appropriate with the Doppler signals received” (col. 7, lines 13-14, the Rock patent). However, there is at least one problem with using such a threshold analysis of the Doppler signals: the wide variety of body shapes and sizes, steady state (i.e., healthy) blood flows, steady state blood pressures, etc. in humankind. Because an AED may be located anywhere that untrained rescuers could operate such a device (e.g., an airplane, a train, a bus, a lobby in a large building, an infirmary, etc.), and the pads of an AED may be placed on a man, a woman, a child, a full-grown adult, an elderly person, someone with a naturally low pulsatile flow, etc., it is difficult, if not impossible, to determine a “universal” threshold that can adequately cover the variety of humans which may or may not need cardiac resuscitation.

Moreover, even in an AED where multiple transducers are used to ensure that one of them captures the artery, the best transducer in a multi-transducer pad might still be offset from the artery by an unknown distance, which means the signals are different compared to the no offset case.

Thus, there is a need for a method and apparatus which can adequately assess the pulsatile flow of the blood of an individual without a priori measurements or knowledge of that particular individual. Furthermore, there is a need for a method and apparatus which can inform an inexperienced and/or untrained user of an AED or any other defibrillation device whether there it is appropriate to defibrillate a patient.

In comparison with the prior art, in which the Doppler signal is analyzed over the entire frequency spectrum, the system and method according to the present invention isolates and analyzes individual frequency bands, thereby recognizing the signal of a weak flow in an individual frequency band, rather than allowing such a signal to be lost in the background noise if the entire frequency spectrum is used. In other words, the signal is better revealed compared to the noise if a small relevant frequency band is used rather than the entire spectrum.

In one aspect of the present invention, a method and apparatus is provided in which a Doppler power spectrogram is first calculated from ultrasound signals backscattered from a body fluid (such as blood in the carotid artery) The power spectra of the individual frequency slices within the calculated Doppler power spectrogram are then calculated. An indicia of the flow behavior of the body fluid is calculated from the power spectrum of each individual frequency slice. Flow behavior may refer to the state of blood perfusion, the state of pulse, the heart beat rate, the flow activity of the blood, and/or the pulsatile activity of the blood. It is contemplated that the present invention may be used on other bodily fluids, as well as other colloidal or emulsion solutions contained in inanimate objects.

In one embodiment, the indicia is a “pulsation index” which is a ratio involving the peak (or peaks) within the frequency slice and the noise within the frequency slice. The pulsation index is an indicator of the pulsatile activity of the blood flow. In another embodiment, an initial measurement of flow behavior is obtained from the patient who is presumably in ventricular fibrillation (VF), and then, after defibrillation, the current flow measurement is normalized to the initial flow measurement to determine whether there is any blood flow. This normalized value is a “flow index”. In other words, a “no pulse” measurement is made during cardiac arrest and then this “no pulse” measurement is subsequently used as a baseline to determine whether current measurements indicate a pulse. The indicia from the individual frequency slices are used to determine whether there is a flow or not. Other indicia of flow behavior are possible in accordance with the present invention.

The present invention is directed to a method and apparatus for ultrasound diagnostics that use selective calculations of the power of a Doppler signal in a plurality of frequency bands of the signal. In exemplary applications, the invention facilitates detection and/or measurements of perfusion, the pulse state of a patient, a heart beat rate, and the like.

In a first aspect of the present invention there is provided an apparatus for ultrasound diagnostics comprising at least one ultrasonic transducer, a generator for exciting the transducer(s), a discriminator of frequency bands of a Doppler signal, and a data processor. In one embodiment, the data processor defines patient's diagnostic information using calculations performed in the frequency bands where, during a cardiac cycle, the power of the Doppler signal has a peak signal-to-noise ratio and/or maximal periodic variations. In one exemplary application, the diagnostic information is obtained using the measurements performed on the patient's carotid artery and includes at least one of perfusion in the artery, the pulse state, and a heart beat rate.

In a second aspect of the present invention there is provided a method for medical ultrasound diagnostics comprising consecutive steps of energizing at least one ultrasonic transducer, selective measuring power of an Doppler signal in a plurality of frequency bands of the signal, and defining diagnostic information. In one embodiment, the diagnostic information is defined using calculations performed in the frequency bands where, during a cardiac cycle, the power of the Doppler signal has the highest signal-to-noise ratio and/or maximal periodic variations and includes at least one of perfusion, the pulse state, and a heart beat rate.

In a third aspect of the present invention there is provided a defibrillation system comprising a defibrillating unit having a controlled source of high-voltage, a controller of the defibrillating unit, an analyzer of diagnostic data, and the inventive apparatus for ultrasound diagnostics. In one exemplary embodiment, the apparatus is used as a source of the patient's diagnostic information for determining whether to defibrillate the patient and for defining parameters of the defibrillating procedure.

Other objects and features of the present invention will become apparent from the following detailed description considered in conjunction with the accompanying drawings. Although fundamental novel features of the present invention as applied to the preferred embodiments shown and described below are pointed out, it will be understood that various omissions and substitutions and changes in the form and details of the embodiments described and illustrated, and in their operation, and of the methods described may be made by those skilled in the art without departing from the spirit of the present invention. It is the intention that the present invention be limited only as indicated by the scope of the claims appended hereto.

The teachings of the present invention will become apparent by considering the following detailed description in conjunction with the accompanying drawings, in which:

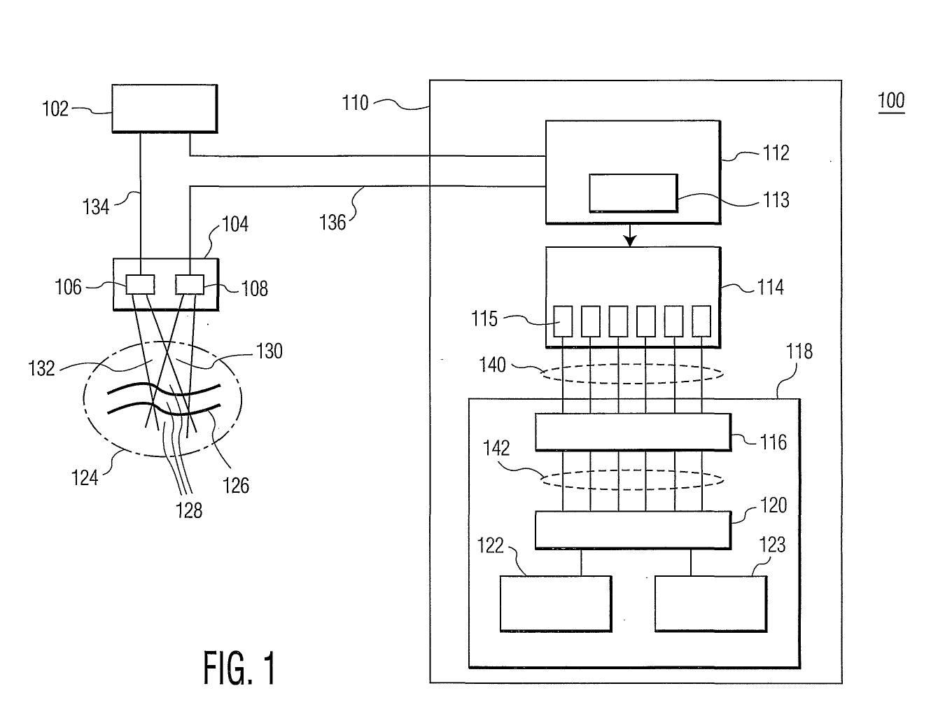

FIG. 1 depicts a block diagram of an exemplary apparatus of the kind that may be used for ultrasound diagnostics in accordance with one embodiment of the present invention;

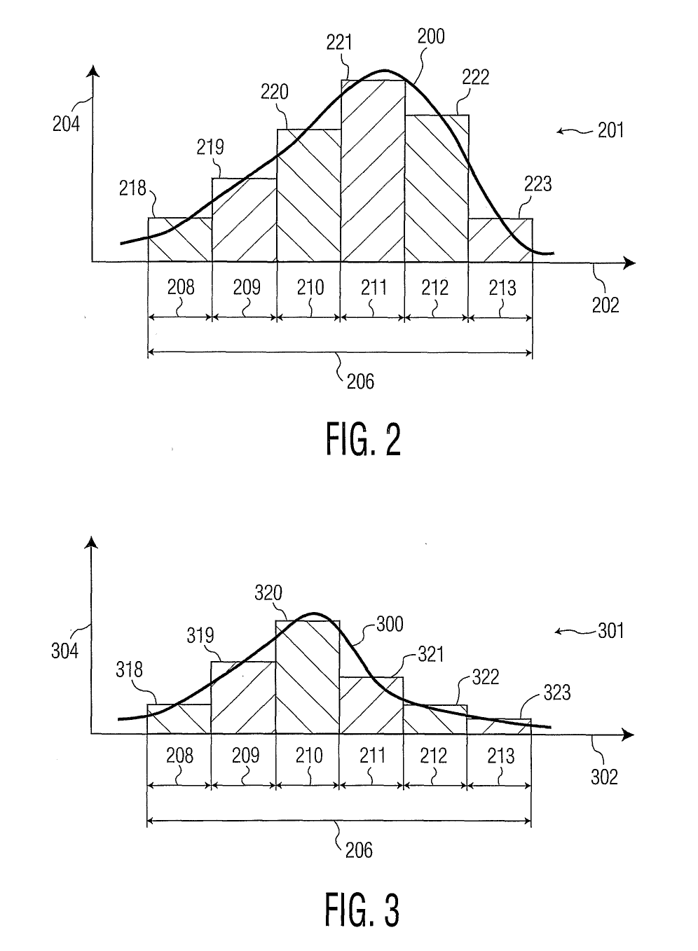

FIG. 2 depicts an exemplary diagram illustrating calculations of the power of a Doppler signal in a plurality of frequency bands of the signal in the apparatus of FIG. 1 during a systolic phase of a cardiac cycle;

FIG. 3 depicts an exemplary diagram illustrating calculations of the power of a Doppler signal in a plurality of frequency bands of the signal in the apparatus of FIG. 1 during a diastolic phase of a cardiac cycle;

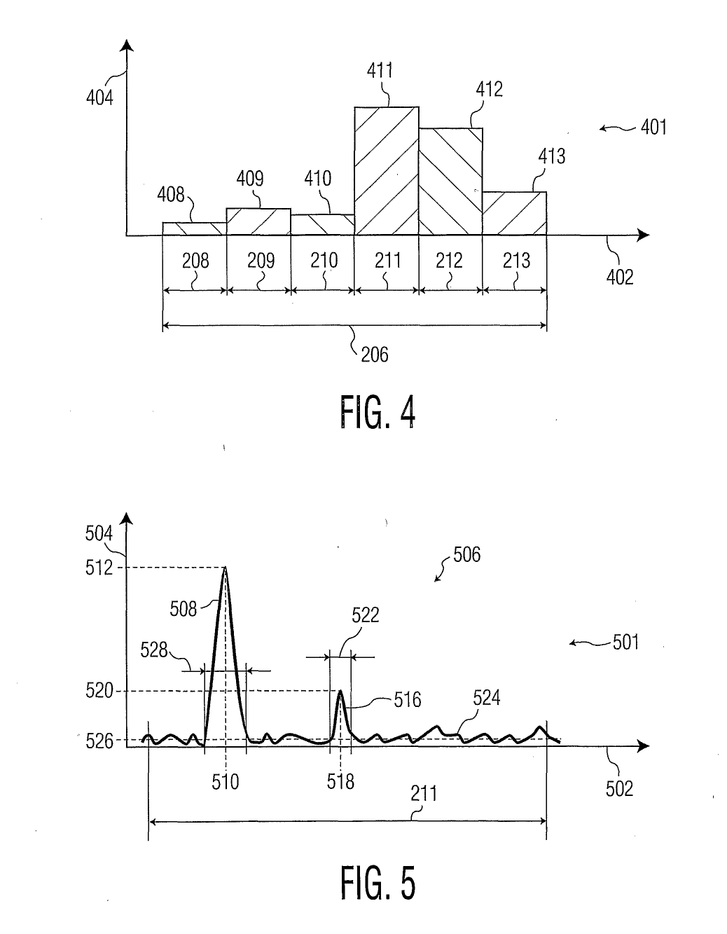

FIG. 4 depicts an exemplary diagram illustrating variations of the power of a Doppler signal in the frequency bands of FIGS. 2-3;

FIG. 5 depicts an exemplary diagram illustrating a result of Fourier analysis of a Doppler signal in a frequency band of FIGS. 2-3;

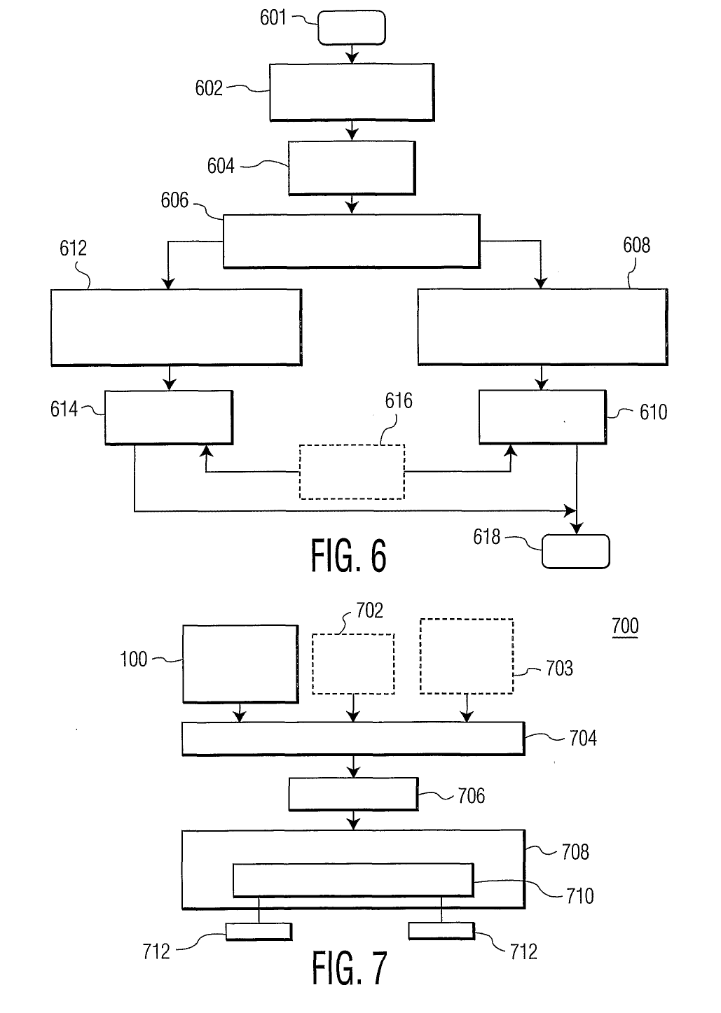

FIG. 6 depicts a flow diagram of one exemplary embodiment of the inventive method for ultrasound diagnostics that may be used during an illustrative procedure of assessing the perfusion or blood pulsing;

FIG. 7 depicts a block diagram of an exemplary defibrillating system including the ultrasound diagnostic apparatus of FIG. 1 in accordance with one embodiment of the present invention;

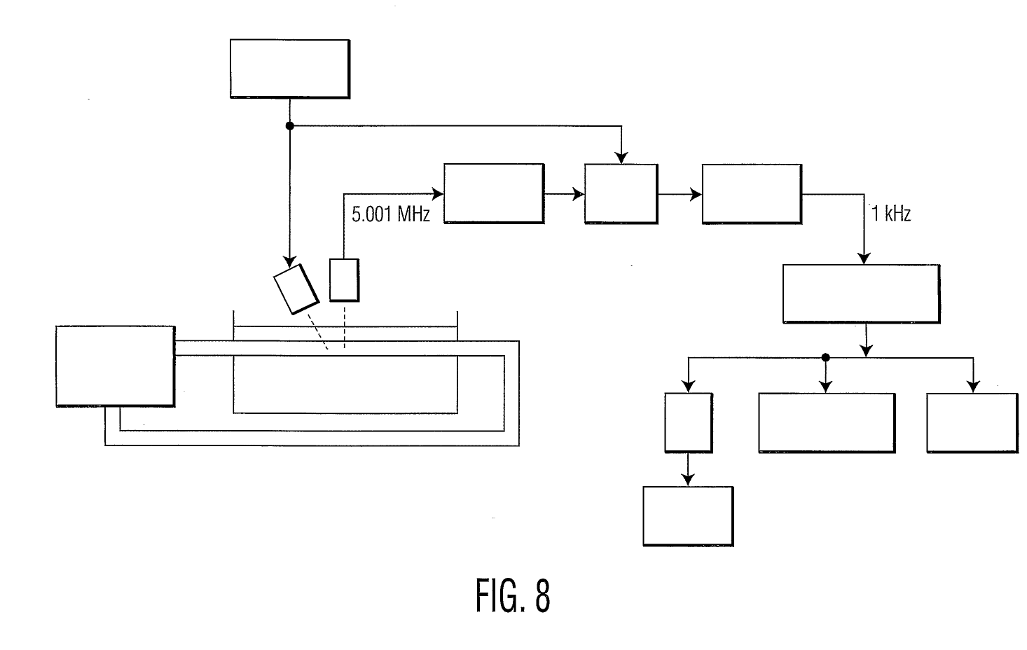

FIG. 8 shows a schematic of an experimental set-up used to test the feasibility of a method and apparatus according to the present invention;

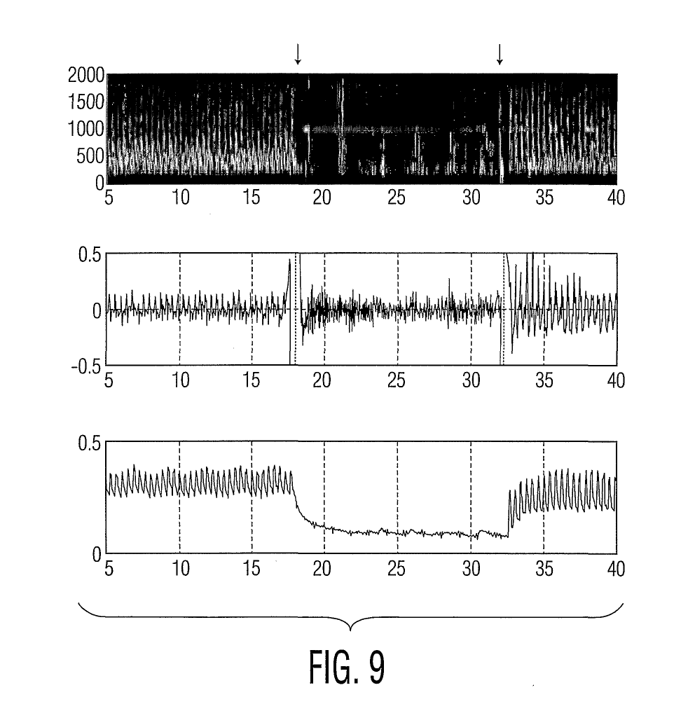

FIG. 9 shows a Doppler spectrogram with the corresponding ECG and arterial blood pressure (ABP) signals taken of a heart in VF using the experimental set-up of FIG. 8;

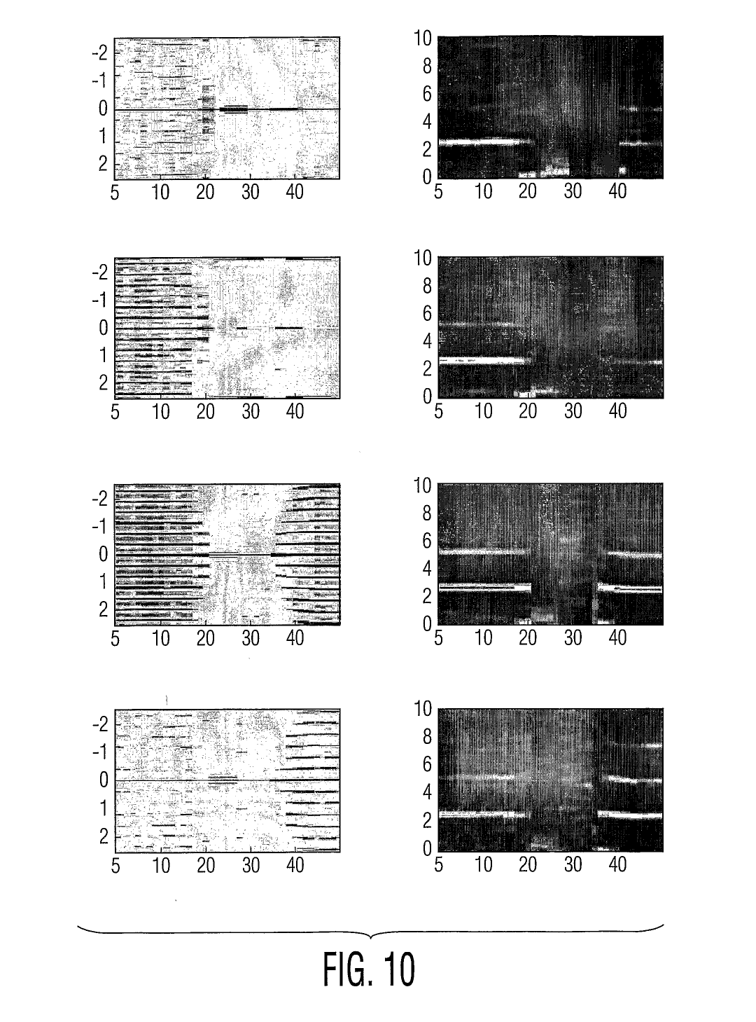

FIG. 10 shows the auto-correlation, and the Fourier Transform of the auto-correlation, of four frequency slices from the Doppler spectrogram of FIG. 9, according to a preferred embodiment of the present invention; and

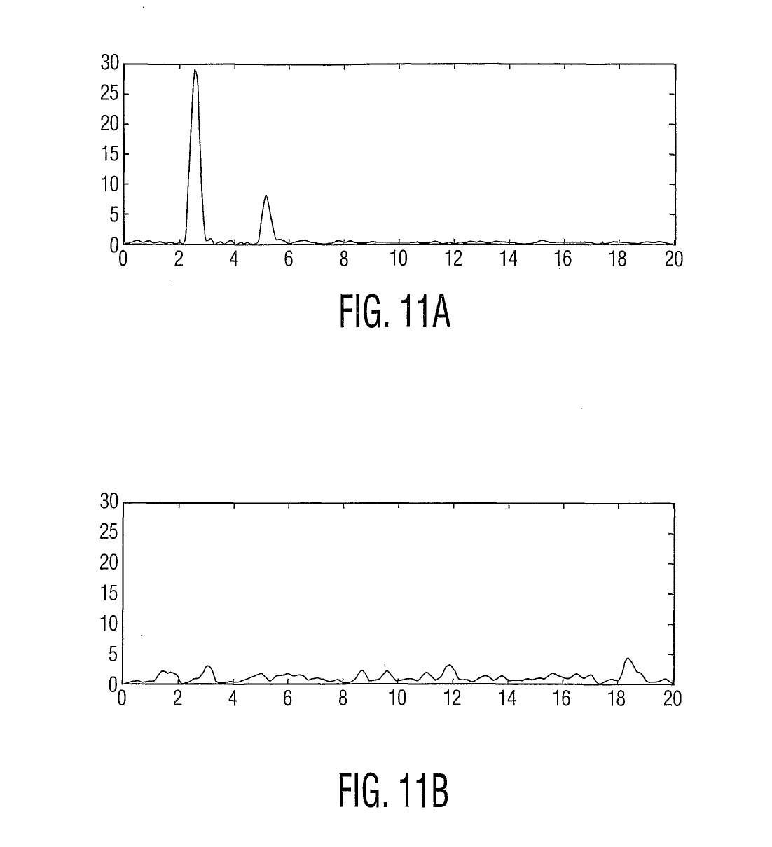

FIG. 11 shows the Fourier Transforms at 10 seconds and at 30 seconds of the auto-correlation of the 1150-1350 Hz frequency slice from FIG. 10, according to a preferred embodiment of the present invention.

Herein, identical reference numerals are used, where possible, to designate identical elements that are common to the figures. The images in the drawings are conventionally simplified for illustrative purposes and are not depicted to scale.

The appended drawings illustrate exemplary embodiments of the invention and, as such, should not be considered limiting the scope of the invention that may admit to other equally effective embodiments.

As discussed above, assessing the pulse state of a patient represents a challenging task, especially in emergencies and during operative procedures, post-operative intensive care, and other life-threatening situations. In such situations, while detecting electrical activity of the heart, an electrocardiogram (ECG) may inadvertently mask the lack of the mechanical activity (i.e., blood pumping functionality) of the heart, thus providing inadequate diagnostic data (leading the caregiver to conclude that there is a pulse) when the heart is in the state of pulseless electrical activity (PEA).

Analyzing the pulsing activity of the heart is problematic if there is weak perfusion, because of the difficulties associated with resolving small variations of a mean (or central) Doppler frequency of the echo signal (i.e., Doppler frequency shifts) at high levels of background spectral noise. Such limitations have a negative impact on the capabilities and clinical efficiency of medical systems using ultrasonic diagnostic information. This is particularly the case when the medical system is intended for use by laymen, such as programmable defibrillators (AED).

The preferred embodiments of the present invention use selective calculations of the power spectrum in each of a plurality of frequency bands of the Doppler spectrogram. The plural frequency bands or slices may comprise the entire frequency spectrum of the Doppler spectrogram, or only two or more preselected slices within the spectrum. In one embodiment, the preselected slices are selected so that their combination will adequately cover as many of the possible indicators of flow behavior in the largest variety of humans (or other subjects). The frequency slices may be of equal or unequal size. Furthermore, the size and location of the frequency slices may be dynamic, i.e., the size and/or location of the frequency slices may change during the analysis of a particular patient.

Any method of ultrasound Doppler can be used with the present invention. The simplest approach is the continuous-wave (CW) Doppler method. In this method, one ultrasound transducer emits a continuous wave signal and another transducer receives the backscattered signal from the region of overlap between the two beams. The received signal, after suitable amplification, is sent to a mixer where signals at the sum and difference frequencies are produced. A low pass filter removes the sum frequency leaving the low frequency base band signal that has a frequency equal to the Doppler frequency. This CW method determines the classical Doppler frequency shift. The drawback of this method is that there is no localization of the signal from blood since the signals from all tissue locations in addition to signals from blood are intrinsically combined.

An alternate method is the pulsed-wave (PW) Doppler technique. In this method, the classical frequency shift is not used. Rather, the phase of the base band signal after demodulation and its change over a repeated set of acquisitions is utilized in reconstructing the Doppler signal. In this method it is possible to select the exact depth at which to analyze the blood or tissue motion. The drawback of this approach is that the electronics required is more complex than the CW case. Also there is the possibility of aliasing if the pulse repetition frequency is not higher than twice the expected Doppler frequency shift. In yet another method, commonly referred to as the Color Doppler technique, the motion of scatterers is determined through a correlation approach. Reflected signals from repeated insonifications are analyzed in order to determine an average motion of scatterers. Although these approaches are mentioned here, any other Doppler method could be used with the present invention, as would be understood by one skilled in the art.

In experiments studying the feasibility of a method and system according to the present invention, the simpler CW method was used. In the preferred embodiment, it is not necessary to know precisely from where the signals were reflected. The backscattered signals are obtained from both the blood flow and all other tissues up to a depth limited by the attenuation of the signal. In order to separate the blood flow from tissue motion, a high pass wall filter was used, based on the assumption that the tissue velocities are of much lower frequency than that of blood flow. The experiments were performed on pigs because their cardiovascular systems are similar to that of humans.

FIG. 8 shows a schematic of the CW experimental set-up, in which a single element transducer (Panametrics, Waltham, Mass.; Model A309S) is excited by an arbitrary waveform generator (Wavetek/Fluke, Everett, Wash.; Model 295), and another transducer identical to the transmit transducer collects the Doppler shifted backscattered echoes. The received signal is amplified using two low noise pre-amplifiers (Minicircuits, Brooklyn, N.Y.; Model ZFL-500LN) each having at least 24 dB of gain, a low noise figure of 2.9 dB, and a rated power output capacity of 5 dBm at 1 dB compression point. The signal after pre-amplification is sent to a mixer (Minicircuits; Model ZP-3MH or other suitable mixers). The mixer also receives a part of the excitation signal from the Wavetek generator at its local-oscillator port. The output of the mixer contains a signal that is the sum and difference of the excitation signal and the received signal. A low pass filter (Minicircuits; Model BLP-1.9) removes the signal at the sum frequency leaving the Doppler signal at the difference frequency to pass through.

Three signals were simultaneously recorded: Ultrasound Doppler, ECG, and Arterial Blood Pressure (ABP). Since it was not a priori possible to estimate the level of Doppler signal from pigs, several additional mixers, filters, and attenuators were made available to allow for flexibility in recording the signals. Filtering (including wall filtering) and amplification of the Doppler signals was performed using a system from Krohn-Hite Corporation (Brockton, Mass.). The Krohn-Hite system was a two-channel tunable filter and amplifier (Model 3382) with a tunable frequency range between 0.1 Hz to 200 kHz. This system had a very sharp cut off frequency (48 dB/octave) which was preferred for the Doppler wall filtering. It also offered considerable flexibility in selecting the gain and filter settings. Each of the channels had a pre-filter gain stage with up to 50 dB gain in 10 dB steps, and a post-filter stage with gain up to 20 dB in 0.1 dB steps. The cut-off frequency could be specified with a resolution of 3 digits. One of the channels in this instrument was used for the high-pass wall filtering and the other for low pass filtering to reduce noise. The high pass cut-off was initially set at 50 Hz but changed to 200 Hz for later experiments. The low pass cut-off was set to 3 kHz.

The Doppler spectrogram created using the data recorded during a typical experiment is shown in FIG. 9. The Doppler spectrogram is essentially a Short Time Fourier Transform (FT) of the Doppler signal and is similar to those displayed on commercial high-end ultrasound systems. Beneath the Doppler spectrogram are shown the corresponding ECG and the ABP signals. The temporal and −3 dB frequency resolutions of the spectrogram were 25 ms and 160 Hz respectively.

FIG. 9 describes the different phases of the cardiac activity during a typical experiment. At the start of the experiment, the heart has its normal beating state. The ECG shows a normal beating rhythm, and the ABP shows the pulsatile nature of the blood pressure in the carotid artery. The corresponding Doppler spectrogram also shows the pulsatile behavior in that the Doppler power moves from the higher frequencies during the systolic phase to the lower frequencies during the diastolic phase. The period of the Doppler spectrogram corresponds to the period of the ABP. At about 18 seconds, an electrical shock is applied to the open heart, which puts the heart in a state of VF. At this point, the ECG loses its normal rhythm and the ABP drops drastically. The corresponding Doppler spectrogram does not show the normal pulsatile behavior seen before the VF. After the animal is in VF for about 15 seconds, a defibrillation shock is applied, causing the heart to recover its beating activity. The ECG returns to the normal rhythm and the ABP increases to a normal rate. The Doppler spectrogram returns to its normal pulsatile state. Although the spectrogram lost its normal pulsatile signature during the period of VF, some activity of the heart, especially at the low Doppler frequencies, could be seen. When the Doppler signal is played on an audio speaker, the pulsatile nature during the initial and the recovery states is apparent, as is the loss of pulsatility during the VF state.

Having created a set of measurements from a series of experiments like that shown in FIG. 9 conducted using the experimental set-up of FIG. 8, various indicia of flow behavior were examined.

As discussed above, in the present invention, the Doppler spectrogram is broken down into two or more frequency slices (i.e., a slice being taken horizontally across the spectrogram shown in FIG. 9) because it is easier to detect pulsatility within a specific frequency band rather than across the total Doppler power spectrum across all frequencies. The specific band in which a pulsatile flow may become apparent depends on many factors, such as the strength of the flow, the Doppler angle, the size of the patient, the normal pulsatile flow of the patient, etc.

In the experiments, four frequency bands were selected for analysis: 225 to 425 Hz, 650 to 850 Hz, 1150 to 1350 Hz, and 1650 to 1850 Hz. These frequency bands were chosen so as to avoid unexpected electrical noise in the recording unit that mostly occurred at 1 kHz, and sometimes at 500 and 1500 Hz. The total Doppler power in these frequency bands was computed as a function of time, which, as mentioned above, is essentially the same as taking a horizontal slice through the spectrogram in FIG. 9. Once the Doppler power within each of the specific frequency bands was calculated, the unbiased auto-correlation of the Doppler power was computed within a 5-second window, which can be seen on the left-hand side of FIG. 10. The time period of 5 seconds corresponds to several cardiac cycles, and is a good trade-off between allowing sufficient time for periodicity estimation and making this period short enough to evaluate as quickly as possible. The auto-correlation function has the property of clearly exposing any periodicity in the signal. The auto-correlation was normalized to have values between −1 and +1. The window was progressively advanced in time (a sliding window) so as to obtain the auto-correlation for the duration of the experiment. The Fourier Transform (FT) of the auto correlation, referred to as the power spectrum, was also computed, and is shown on the right-hand side of FIG. 10. It is expected that during pulsatile activity, the power spectrum would contain a peak at a frequency corresponding to the period of the pulsatile activity. For instance, if the heart rate were 60 beats per minute, the power spectrum would show a peak at a frequency of 1 Hz.

The pulsatile nature of the Doppler power spectrum during the initial and recovery states is readily apparent in the auto correlations shown in FIG. 10. The power spectra during these periods show a peak corresponding to the period of the auto-correlation. It can also be seen that some of the frequency bands (e.g., 1150 to 1350 Hz) expose the periodic nature better than the others.

FIG. 11 shows power spectra in the 1150 to 1350 Hz band obtained from FIG. 10 at two specific time instants. The two time instants correspond to the cases when the 5 sec windows used in the auto correlation ended at 10 and 30 seconds respectively. The former corresponded to the initial state of the heart before fibrillation and the latter to the VF state. It can be seen that during the initial state, the FT showed a peak at a frequency of about 2.58 Hz, which corresponded to a heart rate of 155 beats per minute, the same as that measured by the defibrillator monitoring the ECG signal. In this particular case, a significant second harmonic is also seen at twice the fundamental frequency. During the VF state however the FTs do not show the presence of a strong peak.

It should be noted that the term frequency is used herein differently in different contexts: ultrasound frequency is in the MHz range, the Doppler frequency is in the hundreds of Hz to kHz range, and finally pulse frequency corresponding to the pulsatility of the flow is usually in the range of a few Hz. The different usages should be apparent to one skilled in the art from the context.

The first proposed indicia for flow behavior is directed to measuring the pulsatility of the flow by the periodicity of the Doppler signal. This indicia, called the “pulsation index”, is a ratio of the power in a peak in the power spectrum of a frequency slice (e.g., FIG. 11) to the power in the total power of the power spectrum of the frequency slice (or just the background of the total power spectrum, i.e., the spectrum excluding the peak or peaks).

When finding the pulsation index according to a preferred embodiment of the present invention, the Doppler power in several frequency bands is computed as a function of time, followed by the computation of the auto-correlations and power spectra, as has been described above. A peak-searching algorithm then determines the frequency at which the power spectrum is a maximum. The fraction of the total power contained within a narrow band around this frequency peak is determined. For the case of normal pulsatile flow, one would expect that a significant portion of the total power would be present in this narrow band whereas that would not be the case when pulsatile flow is absent.

A priori assumptions based on physiology could be used to restrict the search space for the location of the peak in the power spectrum. For instance, for the data recorded from pigs, it could be assumed that during normal flow in the carotid, the heart rate would be between 40 and 240 beats per minute. Thus the algorithm would search for the global peak between 0.67 and 4 Hz. The bandwidth of the narrow band is determined by the total time duration of the auto-correlation. Since the auto-correlation was computed over a lag time of T=5 seconds, the useful bandwidth was taken to be 80% of 4/T=0.64 Hz (80% would capture most of the main lobe width). There are a few cases where no maximum were to be found within this range. In such cases, the algorithm would set the computed index to be zero.

Although many possible pulsation indices are possible in accordance with the present invention, three possible pulsation indices will be considered herein. In each case, the pulsation index takes values ranging between 0 and 1, with higher values expected for the flow case and lower values for the no flow case.

The first pulsation index is the ratio of the power in the narrow band around the frequency peak to the total power in the signal over all the frequencies.

The second pulsation index is the ratio of the sum of total power in the narrow bands around the peak frequency and at twice the peak frequency (referred to as the second harmonic frequency) to the total power in all frequencies. This measure accounts for the fact that the pulsatile signal is not sinusoidally periodic, and consequently can contain additional harmonics. For simplicity, only the second harmonic is included and the higher order harmonics are not considered.

The third pulsation index is the ratio of the power in the narrow band around the peak frequency to that of the total power excluding the second harmonic. This is similar to the first measure except that the denominator excludes the power in the second harmonic.

While all three indices quantify the periodic behavior in the Doppler power, a heuristic analysis can be invoked to prefer one over the other two. In this analysis, it is assumed that the flow case contains a peak at a fundamental frequency and a smaller peak at the second harmonic, whereas the no flow case is essentially noise for which the power spectrum is essentially low and constant at all frequencies.

For the no flow case, the second pulsation index would be about twice that of the first pulsation index, since twice the amount of noise is present in the numerator. For the flow case, the second pulsation index would be less than twice that of the first pulsation index, since the second harmonic is of smaller magnitude than the fundamental frequency. Thus, there would be a larger separation in the index values between the two cases for the first pulsation index than for the second pulsation index. Therefore, if the task is to discriminate the flow case from the no flow case, the first pulsation index is preferred over the second pulsation index.

The difference between the first and third pulsation indices only lies in the denominator, i.e., the absence of the second harmonic contribution in the denominator of the third pulsation index. For the no flow case, removing the second harmonic would only remove a small contribution in the denominator leaving the index unaffected. Thus the two indices would have similar values. However, in the flow case, removing the contribution from the second harmonic would lead to a significant reduction in the denominator, and would thus increase the value of the third pulsation index closer to unity than the first pulsation index. Thus, the discrimination between the flow and no flow case would be larger in the case of the third pulsation index. In this heuristic analysis, the third pulsation index is the most preferred among the three indices.

According to one embodiment of the present invention, the pulsation index is computed for several slices, and the maximum among the pulsation index values of all the frequency slices is used to determine whether there is a flow or not. Because the frequency band that best captures the pulsatility information depends on several factors, such as the Doppler frequency, the Doppler angle, and the blood flow conditions (e.g., the condition of the patient's artery, the normal pulsatile flow of the patient, etc.), it is not possible to select a priori the optimal frequency band. Thus, in this embodiment, it is assumed that the maximum pulsation index value would be the most optimal band for finding whether a pulse is present. However, in other embodiments of the present invention, the pulsation index values among the various frequency slices can be manipulated differently in order to determine whether a flow is present.

The second proposed indicia for flow behavior is directed to measuring the overall flow, regardless of whether it's pulsatile or steady. It is based on the fact that the overall Doppler signal in a specific frequency band should be high for the flow case and low for the no flow case. This indicia, called the “flow index”, would be equivalent to the actual brightness of the pixels in a Doppler spectrogram shown on the display of a conventional ultrasound system. Since the Doppler signal could vary largely from one patient to another, such a quantity would require appropriate normalization. It is preferable to perform this normalization based on the same patient.

One possible way for accomplishing this is to use the fact that many patients at the time of intervention with an AED would already be in a state of VF, i.e., in a state where there is no flow. Thus, one could use this time period to obtain a Doppler signal value and establish this Doppler measurement as the “definition” of the no flow situation. Subsequently, after defibrillation, one could compare the current Doppler power measurements with the prior no flow situation in order to determine whether there is any flow. In one preferred embodiment of an AED using this flow index, the 90^{th} percentile point of the Doppler power spectrum in a particular frequency band is initially computed (while the patient is presumably in VF) over a window of 5 seconds. This initial “no flow” measurement is then used to normalize all future measurements: this normalized measure is the flow index. As can be seen in this example, the flow index is an indicator of the overall flow and is different in nature from the pulsation index. It should be noted that this quantity should be computed only if the AED determines that the patient at the time of intervention is in a state of VF. Obviously, this measure could be used in determining the presence of a post-defibrillation PEA.

As in the preferred embodiment using the pulsation index, the flow index value for several frequency slices is computed and the maximum among the slices is selected as the flow index. In other embodiments, the flow index of several or all the frequency slices could be used. When there is a flow, the flow index should be significantly larger than unity, whereas for the PEA case the flow index should be closer to unity. The choice of the 90^{th} percentile value is somewhat arbitrary, but the maximum value is very susceptible to noise, and the mean value does not exploit the fact that the flow during systolic phase is higher than the mean flow during a cardiac cycle.

The indicia of flow behavior used in the preferred embodiments (i.e., the pulsation index and the flow index) have many advantages over other measurements used to determine flow behavior. Although a measure such as the mean Doppler frequency shift over the entire Doppler spectrogram has the potential to perform well in determining pulsatility, the fact that, for an AED, the flow conditions (flow velocity, angle of flow, etc.) of the patient are not exactly known means the expected behavior of the mean Doppler frequency shift is also unknown. The indicia for flow behavior directed to pulsatile flow disclosed herein do not suffer from this pitfall, and thusly, appear to be more robust measures for pulse state assessment. However, it is possible for the mean Doppler shift within each frequency slice to be used in accordance with the present invention.

As another example of the advantages of the pulsation index, consider using the periodicity of the cross correlation between the Doppler signal and the ECG signal as a measurement of pulsatile flow. When the patient is in a state of pulseless electrical activity (PEA), such a cross-correlation would still show a significant level of periodicity, although lower than for the normal flow case, because the ECG remains periodic even while the Doppler signal is not. One could simply use the value of the cross correlation as a measure of pulsation index, but this has disadvantages. Because the actual value of the cross correlation would depend on the shape of the ECG signal and the Doppler signal, and since the ECG signal in general could assume a variety of shapes depending on the heart condition of the patient, it would be difficult to a priori predict its expected shape, and set a threshold for determining whether there is good correlation with the Doppler signal or not.

Another advantage of the indicia of flow behavior directed to pulsatile flow according to the preferred embodiments of the present invention is that they rely solely on the Doppler signal, and do not rely on any correlation with other signals (e.g., ECG), and hence can be used in stand-alone pulse detection systems.

While the indicia of flow behavior used in the preferred embodiments (i.e., the pulsation index and the flow index) are useful indicators in their own right, it is also possible that these (and other) indicia could be combined together and used in automatically assessing these and other aspects of flow behavior.

The exemplary pulsatile indices used in the preferred embodiments are based on a search for a sinusoidal type of periodicity. However, because the Doppler signal is not sinusoidally periodic, there are harmonics in the power spectrum, which can affect the value of the pulsation index. To avoid this, the second harmonic was removed from the denominator of the third pulsation index. In future embodiments, a more appropriate type of analysis, such as wavelet analysis, could be used to detect the non-sinusoidal periodicity of the Doppler signal.

A primary advantage of a method and system according to the present invention is the ability to adequately assess the flow of a body fluid, such as blood, of an individual without a priori measurements or knowledge of that particular individual. This is of great use in AEDs or other defibrillation devices which require an inexperienced and/or untrained user to determine whether it is appropriate to defibrillate a patient. The robustness of using frequency slices and indicia of flow behavior according to the present invention make the inventive method and system appropriate for defibrillation systems such as AEDs where the possible variation in placement of the ultrasound sensors, the variation in direction of the flow in relation to the sensors, the wide variety of possible patient body shapes and sizes, the wide variety of different “normal” (i.e., healthy) blood flows, the wide variety of different “normal” (i.e., healthy) blood pressures, etc. make it impossible to have too many a priori assumptions about the measurements.

Moreover, the method and system according to the present invention is not limited to human and/or animal care or diagnosis. For example, the method and system could be used for the analysis of any fluid mass which can be measured by ultrasound Doppler, including, but not limited to, the analysis of underground fluid deposits or streams, the analysis of pipeline flow and/or dynamics, or the analysis of practically any fluid dynamic system.

Having described the inventive method in general, and described various embodiments of indicia of flow behavior, an exemplary embodiment of a system according to the present invention will now be described.

FIG. 1 depicts a block diagram of an exemplary apparatus 100 of the kind that may be used for ultrasound diagnostics in accordance with one embodiment of the present invention. In one exemplary application, the apparatus 100 can perform assessment (e.g., detection and/or measurements) of perfusion and/or the pulse state of a patient. Herein the term “perfusion” refers to blood flow in a blood vessel (e.g., carotid artery) or a tissue. In other applications, the apparatus 100 may be used as a component in resuscitation systems and defibrillators, monitors and detectors of weak heart beat (e.g., fetal heart beat), among other medical diagnostic and clinical systems. Additionally, the apparatus 100 may also be used in non-medical systems for measuring, for example, flow or pulsatile activity of colloidal and emulsion solutions.

In one embodiment, the apparatus 100 comprises a generator 102, at least one ultrasonic transducer 104 (one transducer 104 is shown), and a data processor 110. In alternate embodiments, the transducers 104, together, form an array that typically is disposed upon an application pad (not shown), and the transducers may additionally be time multiplexed. Such arrays are disclosed, for example, in the previously mentioned Rock patent.

In the depicted embodiment, the transducer 104 comprises a transmitter 106 and a receiver 108. In this embodiment, the generator 102 is generally a source of a continuous wave (CW) radio frequency (RF) signal (e.g., 1-10 MHz). In operation, the generator 102 via interface 134 activates (or excites) the transmitter 106 to emit ultrasound (illustratively shown as a beam 132) propagating in a portion 124 of the body of a patient located beneath the transducer. The receiver 108 collects, within an aperture 130, an acoustic echo signal (i.e., scattered ultrasound), transforms the echo signal into an electrical signal and transmits, via interface 136, to the data processor 110. The transmitter 106 and receiver 108 are positioned such that the beam 132 and aperture 130 overlap in a region 128 of a large blood vessel 126, such as a carotid artery, and the like.

In an alternate embodiment, the apparatus 100 may comprise the transducer 104 capable of operating as a transmitter when RF power is ON, or a receiver when the RF power is OFF, respectively. In this embodiment, the generator 102 produces pulsed RF power (PW) having duration of an ON time interval of about 0.2 to 20 microseconds and a duty cycle in a range of about 0.2 to 20%.

In one exemplary embodiment, the data processor 110 comprises a signal acquisition module 112, a frequency band discriminator 114, and a signal analyzer 118 including a processing module 120, a perfusion detector 122, and a pulse state detector 123. Components of the data processor 110 may be reduced to practice in a form of electronic hardware, a computer program (i.e., software), or both. Alternatively, portions of signal processing performed by the module 110 may also be accomplished using a remote processor (not shown). Moreover, in another embodiment, the analysis may be performed in the analog, rather than the digital, domain, e.g., frequency band discriminator 114 could be replaced with an analog filter bank, data processor 110 could comprise a correlator, etc., as would be known to one of ordinary skill in the art.

The signal acquisition module 112 acquires the echo signal and defines a Doppler signal. Herein, the term “Doppler signal” relates to a signal that is proportional to a frequency shift between the incident ultrasound and the echo signal. Illustratively, the module 112 includes frequency converters of the echo signal, analog and digital filters, memory devices, computer processors, and other means conventionally used for data acquisition and digital signal processing. One filter may be a high frequency pass filter that suppresses the echo originated in the region 128 by stationary or slowly moving objects, such as tissues, walls of the blood vessel 126, the like. In one embodiment, the module 112 stores in a memory 113 in a digital format the Doppler signal that has been acquired during at least one time interval ΔT₁ having duration of about 2 to 20 sec (preferably 5-10 sec). In this embodiment, from the memory 113, the stored digitized Doppler signal may be provided for further processing to the frequency band discriminator 114 in a form of consecutive data banks each relating to a time segment ΔT₂ having duration of about 10 to 100 msec (e.g., 40 msec).

In one embodiment, the frequency band discriminator 114 comprises a plurality (e.g., 4 to 10) of band pass filters 115 (six filters 115 are shown), which selectively decompose the Doppler signal in a plurality of sampling signals 140. Each sampling signal 140 has a frequency range that represents a portion of a pre-selected frequency range of the Doppler signal, wherein such ranges do not overlap. Hereinafter, the terms “frequency range” and “frequency band” are used interchangeably. Together, frequency ranges of the sampling signals 140 comprise the frequency range of the decomposed Doppler signal or a portion of it.

The band pass filters are selectively calibrated to have the same coefficient of amplification that may be either greater or smaller than 1. As such, the sampling signals 140 preserve instant spectral power distribution of the Doppler signal as provided by the signal acquisition module 112 and, therefore, power of each sampling signal is proportional to the power of the Doppler signal in the frequency range of the respective sampling signal 140. In the depicted embodiment, an output of each band pass filter 115 is illustratively coupled to a respective input of the power metering unit 116. In an alternate embodiment (not shown), such outputs may be multiplexed (e.g., time multiplexed) and be coupled to the power metering unit 116 using a single transmission line.

The power metering unit 116 selectively calculates the power of each of the sampling signals 140 and outputs to the processing module 120 a plurality of signals 142 each representing the power of the respective sampling signal as averaged for duration of the time segment ΔT₂. One skilled in the art will readily appreciate that the signals 142 may also be multiplexed (e.g., time multiplexed) and coupled to the processing module 120 using a single transmission line.

To assess the perfusion, in one exemplary embodiment the processing module 120 selectively computes a measure of periodicity of the Doppler signal selectively in each frequency band of the signal using, e.g., a ratio of the power of the Doppler signal to baseline noise. A peak value of the ratio and the data identifying the frequency band having such a ratio are transmitted to the perfusion detector 122. In the perfusion detector 122, the computed peak ratio is compared with pre-determined settings to assess a velocity of the blood flow in the examined blood vessel (e.g., carotid artery). Data relating to a specific pattern of the spectral power distribution of the Doppler signal may also carry additional diagnostic information regarding mechanical activity of the patient's heart and, as such, be preserved, e.g., in a memory of the signal analyzer 118 or, alternatively, data processor 110.

To assess a measure of periodicity of the Doppler signal and, as such, the state of the pulse, in one exemplary embodiment the processing module 120 defines the output signal 142 that, during the time period ΔT₁, experiences greater variations (i.e., maximal periodic variations) in the power than other signals 142. Variations in the Doppler power correspond to transitions between systolic and diastolic phases of a cardiac cycle (discussed in detail in reference to FIGS. 2-4 below). One computational technique includes auto-correlation analysis of the power of the Doppler signal over a pre-determined time interval to determine if an auto-correlation function has periodically spaced peaks identifying a pulsatile activity of the heart. Results of the auto-correlation analysis are transmitted to the pulse state detector 123. In the pulse state detector 123, the intensity of blood pulsing may be assessed using, for example, a pulsation index PI (discussed in detail in reference to FIG. 5 below), and the like measures of the periodicity. The computed value of the selected measure of periodicity may be compared with pre-determined settings and/or thresholds to define and assess the state of the pulse in the blood vessel 126.

In one embodiment, the processing module 120 collects output signals 142 during a period of time that encompasses several cardiac cycles. Illustratively, the processing module 120 may acquire the signals 142, in a form of blocks of data each relating to the segment ΔT₂, for duration of the time interval ΔT₁ extending over several cardiac cycles and selectively process each such a block of data. The processing module 120 may utilize computational techniques known to those skilled in the art, such as algebraic and Boolean logic operations, spectral analysis, Fourier analysis (e.g., Fast Fourier transform (FFT) analysis), correlation analysis, and other signal processing techniques.

FIG. 2 depicts an exemplary diagram illustrating calculations of the power of a Doppler signal in the apparatus of FIG. 1 during a systolic phase of a cardiac cycle. More specifically, a graph 201 depicts an exemplary spectral power distribution (y-axis 204) of the Doppler signal 200 versus frequency (x-axis 202). In apparatus 100, power of the Doppler signal 200 is selectively measured in pre-determined frequency ranges (illustratively, six frequency ranges 208-213 are shown) that, together, represent a frequency range 206 of the Doppler signal. In one embodiment, each such frequency range has a bandwidth of about 100 to 500 Hz, e.g., 200 Hz. Levels of the power of the Doppler signal 200 in the frequency ranges 208-213 are denoted herein using numerals 218-223. In one embodiment, each of levels 218-223 corresponds to a respective output signal 142 of the power metering unit 116 as measured during one of the time segments ΔT₂ of the systolic phase.

FIG. 3 depicts an exemplary diagram illustrating calculations of the power of a Doppler signal in the apparatus of FIG. 1 during a diastolic phase of the cardiac cycle. More specifically, a graph 301 depicts an exemplary spectral power distribution (y-axis 304) of a Doppler signal 300 versus frequency (x-axis 302). Power levels 318-323 correspond to the outputs signals 142 of the power metering unit 116 as measured, in the frequency ranges 208-213, as measured during one of the time segments ΔT₂ of the diastolic phase.

FIG. 4 depicts an exemplary diagram illustrating variations (i.e., difference between maximal and minimal values) in the power of a Doppler signal in the frequency bands 208-213 of FIGS. 2-3 between the systolic and diastolic phases of the same cardiac cycle. Such variations correspond to pulsatile (i.e., mechanical) activity of the patient's heart. More specifically, a graph 401 depicts an absolute value of such a difference (y-axis 404) in the Doppler power versus frequency (x-axis 402). In the depicted embodiment, the difference 411 in the Doppler power is illustratively greater in the frequency band 211 than in any other frequency band in the frequency range 206 of the Doppler signal. When the ultrasonic measurements are performed on the same patient at other state of the patient's cardiac activity or upon different patients, power variations between the systolic and diastolic phases may attain the maximal value in various frequency bands. Generally, when blood flow in the blood vessel is slow due to, for example, a weak heart, the pulsatile activity may be detected in lower frequency bands. Oppositely, the pulsatile activity may be better assessed in the higher frequency bands when perfusion is strong, as in case of a healthy individual.

FIG. 5 depicts an exemplary diagram illustrating a result of Fourier analysis of the power of the Doppler signal in a frequency band of FIGS. 2-3. More specifically, a graph 501 illustratively depicts an amplitude (y-axis 504) of an auto-correlation function 506 of the power versus frequency (x-axis 502) in the frequency band 211. Typically, the auto-correlation function 506 comprises a main peak 508 having a bandwidth 528 centered at a frequency 510, a second harmonic peak 522 having a bandwidth 522 centered at a frequency 518, and a noise floor 524 having an average level 526. The peak 522 is originated by non-harmonic components in the heart rhythm and, typically, has a height 520 that is 3-10 times smaller than a height 512 of the main peak 508. In assessment of the pulsing activity, the peak 522 may computationally be excluded from calculations. In one embodiment, assessment of the FT of the auto-correlation function 506 includes calculating the pulsation index PI that is defined as a ratio of the power in the bandwidth 528 to the power in the frequency range 206 excluding the power in the bandwidth 522.

It should be noted that auto-correlation functions of the signals relating to variations in the power of the Doppler signal in the other bands of the frequency range 206 (i.e., bands 208-210 and 212-213) may have a pattern similar to that in the frequency band 211. However, corresponding auto-correlation functions comprise either lower correlation peaks, or higher noise levels, or both. As such, calculations performed upon analysis of the Doppler power in the frequency band 211 provides high accuracy of assessing the mechanical activity of the patient's heart.

FIG. 6 depicts a flow diagram of one exemplary embodiment of the inventive method for ultrasound diagnostics. The method may be reduced to practice, e.g., using the apparatus of FIG. 1 for performing an illustrative procedure of detecting blood perfusion and/or the pulse state of a patient. To best understand the invention, the reader should simultaneously refer to FIGS. 1-5.

The method starts at step 601 and proceeds to step 602. At step 602, at least one ultrasonic transducer 104 is activated to emit ultrasound towards the blood vessel 126 (e.g., carotid artery) and collect the echo signal scattered in the region 128 of the body of a patient. The ultrasonic echo signal is converted to the electrical format and transmitted to the data processor 110. At step 604, the echo signal is acquired for duration of the time interval ΔT₁, digitized, and stored in a memory, as discussed above in reference to FIG. 1. The time interval ΔT₁ typically encompasses several (e.g., 3-6) cardiac cycles. Alternatively, the time interval ΔT₁ may have a pre-determined duration. At step 606, spectral power distribution of the Doppler signal is defined in a plurality of discrete frequency bands and averaged within time segments ΔT₂ of the time interval ΔT₁. At step 608, a frequency band having, during a cardiac cycle, maximal periodic variations of the Doppler power is defined and, at step 610, the pulse state of the patient is calculated, as discussed in detail in reference to FIGS. 4-5. At step 612, a frequency band having, during a cardiac cycle, a peak ratio of the Doppler power to baseline noise is defined and, at step 614, the perfusion is calculated as discussed above in reference to FIG. 1. At an optional step 616, data collected using simultaneously operating electrocardiograph (ECG system) may be used when, e.g., the method is reduced to practice in a defibrillating system, as discussed in reference to FIG. 7 below. In this case, timing of the ECG data should be conventionally adjusted for a time lag between the ECG and ultrasound spectrograms. In one embodiment, steps 608, 610, 612, 614, and 616 may be performed substantially simultaneously. Upon completion of steps 610 and 614, the method proceeds to step 618 where the method ends.

FIG. 7 depicts a block diagram of an exemplary programmable defibrillating system 700 in accordance with one embodiment of the present invention. Illustratively, the defibrillating system 700 comprises the ultrasound diagnostic apparatus 100 of FIG. 1, an optional ECG system 702, an optional blood pressure monitor 703, an analyzer 704 of diagnostic information, a defibrillating unit 708, and a programmable controller 706 of the defibrillating unit.

The apparatus 100 provides to the analyzer 704 diagnostic information relating to the mechanical activity of the heart and including at least one of the perfusion and the pulse state of a patient (e.g., the pulsation index PI). Ultrasonic diagnostic information may be obtained using the measurements performed on the patient's carotid artery. Such information may additionally be used in diagnosing, in real time, the state of blood supply to the brain of the patient.

In one embodiment, the ECG system 702 and the apparatus 100 acquire the diagnostic data simultaneously. In this embodiment, the signal related to the spectral distribution of the power of the Doppler signal (discussed in reference to FIGS. 1-5 above) may further be cross-correlated with an ECG signal. Such correlation may further increase accuracy and reliability of interpreting the diagnostic information by the analyzer 704.

In a further embodiment, each of the signals 142 may be coupled to the analyzer 704 where the signals 142 are selectively cross-correlated with the ECG signal to provide most accurate assessment of the perfusion, whereas the ABP monitor may be used as a source of data characterizing an overall state of mechanical activity of the heart. Alternatively, the analyzer 704 may use only the diagnostic information provided by the apparatus 100.

It should be noted, however, that the ECG signal corresponds to the electrical activity of the heart. Exclusive use of the ECG diagnostics in the system 700 may result in masking the lack of the mechanical activity (i.e., blood pumping functionality) of the patient's heart by the pulseless electrical activity (PEA) of the heart and, as such, cause erroneous clinical decisions.

The analyzer 704 performs analysis of collected information to determine whether to defibrillate the patient and define parameters of a defibrillation procedure. In operation, the analyzer 704 outputs the results of the analysis to the programmable controller 706 that configures the defibrillating unit 708 comprising a controlled source 710 of high voltage and application electrodes 712 (two electrodes 712 are shown) for executing the procedure.

In illustrative embodiments discussed in reference to FIGS. 1 and 7 above, many portions of apparatus 100 and system 700 are available in medical ultrasound and defibrillation systems and application specific integrated circuits (ASICs) available from Koninklijke Philips Electronics N.V. of Eindhoven, Netherlands.

Thus, while there have been shown and described and pointed out fundamental novel features of the present invention as applied to preferred embodiments thereof, it will be understood that various omissions and substitutions and changes in the form and details of the devices described and illustrated, and in their operation, and of the methods described may be made by those skilled in the art without departing from the spirit of the present invention. For example, it is expressly intended that all combinations of those elements and/or method steps which perform substantially the same function in substantially the same way to achieve the same results are within the scope of the invention. Substitutions of elements from one described embodiment to another are also fully intended and contemplated. It is the intention, therefore, to be limited only as indicated by the scope of the claims appended hereto.

1. A method for detecting and/or measuring, using an ultrasound device, flow behavior of a fluid within a subject, comprising the steps of:
determining a total Doppler power for each of a plurality of frequency slices as a function of time, wherein said total Doppler power is calculated from an ultrasound signal backscattered from the fluid within the subject;
determining power spectra from the determined total Doppler power whereby each of the plural frequency slices has a power spectrum over the frequencies within that frequency slice; and
calculating an indicia of flow behavior of the fluid within the subject for each frequency slice;
whereby flow behavior is measured and/or detected using at least one of the calculated indicia of flow behavior of each frequency slice.

2. The method of claim 1, wherein flow behavior comprises at least one of a state of blood perfusion, a state of pulse, a heart beat rate, and/or flow and/or pulsatile activity of a colloidal or emulsion solution.

3. The method of claim 1, wherein the step of determining the power spectra uses at least one of spectral analysis, Fourier analysis, correlation analysis, an averaged periodogram estimate, parametric methods, and/or auto-correlation analysis of the Doppler signal.

4. The method of claim 1, wherein the step of determining power spectra comprises the steps of:
determining an auto-correlation of each of the plural frequency slices over a sliding window of time; and
determining power spectra from the determined auto-correlations.

5. The method of claim 4, wherein the sliding window used in the step of determining the auto-correlation has a length in a range of about 2 to about 20 seconds.

6. The method of claim 4, wherein the sliding window used in the step of determining the auto-correlation has a dynamically changing length.

7. The method of claim 4, wherein the subject is a human or an animal, and wherein the sliding window used in the step of determining the auto-correlation has a length selected in order to cover at least two periods of pulsation of the fluid being detected and/or measured in the human or animal.

8. The method of claim 1, further comprising the step of:
selecting the frequency slice with the indicia of flow behavior having a maximum value, wherein the selected maximum value is used to measure and/or detect the flow behavior.

9. The method of claim 1, wherein the indicia of flow behavior comprises a pulsation index, said pulsation index comprising a ratio involving at least one of one or more peaks in the power spectra of the frequency slice and the total power in the power spectra of the frequency slice.

10. The method of claim 9, wherein the pulsation index comprises a ratio of the power in the greatest peak in the power spectra of the frequency slice and the total power in the power spectra of the frequency slice.

11. The method of claim 9, wherein the pulsation index comprises a ratio of the power in the greatest and the second greatest peak in the power spectra of the frequency slice and the total power in the power spectra of the frequency slice.

12. The method of claim 9, wherein the pulsation index comprises a ratio of the power in the greatest peak in the power spectra of the frequency slice and a quantity comprising the total power in the power spectra of the frequency slice minus the power in the second greatest peak in the power spectra of the frequency slice.

13. The method of claim 1, further comprising the steps of:
obtaining an initial value for a measurement of flow behavior in at least one frequency slice; and
obtaining a later value for the measurement of flow behavior in the at least one frequency slice;
wherein the step of calculating the indicia of flow behavior comprises the step of:
normalizing said later value with said initial value in order to obtain a flow index which comprises the indicia of flow behavior.

14. The method of claim 13, wherein the step of obtaining the initial value is performed while the subject is in ventricular fibrillation, and the step of obtaining the later value is performed after the subject has been defibrillated.

15. The method of claim 13, wherein the measurement of flow behavior is the mean, peak, or 90^{th} percentile value over a window of time of the power spectrum of the at least one frequency slice.

16. The method of claim 1, wherein each of the plural frequency slices has the same bandwidth.

17. The method of claim 1, wherein at least one of the plural frequency slices has a bandwidth in a range of about 100 Hz to about 400 Hz.

18. The method of claim 1, wherein at least one of the plural frequency slices has a bandwidth which is dynamically changing.

19. The method of claim 1, wherein said method steps are performed in a defibrillator.

20. The method of claim 19, wherein said defibrillator comprises an Automated or Semi-Automated External Defibrillator (AED).

21. The method of claim 1, wherein the subject is a human, an animal, another animate object, and/or an inanimate object.

22. A method for detecting, using an ultrasound device, a pulsatile flow of a fluid within a subject, comprising the steps of:
determining a total Doppler power for each of a plurality of frequency slices as a function of time, wherein said total Doppler power is calculated from an ultrasound signal backscattered from the fluid within the subject;
determining power spectra from the determined total Doppler power whereby each of the plural frequency slices has a power spectrum over the frequencies within that frequency slice;
calculating a pulsation index for each frequency slice, said pulsation index comprising a ratio involving at least one of one or more peaks in the power spectra of the frequency slice and the total power in the power spectra of the frequency slice; and
determining whether there is a pulsatile flow of the fluid within the subject by comparing each of the calculated pulsation indices to a predetermined threshold value, wherein there is a pulsatile flow if any of the calculated pulsation indices exceeds the predetermined threshold value.

23. The method of claim 22, wherein said method steps are performed in a defibrillator.

24. The method of claim 23, wherein said defibrillator comprises an Automated or Semi-Automated External Defibrillator (AED).

25. A method for detecting, using an ultrasound device, whether there is a flow of a fluid within a body of a subject who has recently experienced ventricular fibrillation, comprising the steps of:
obtaining at least one initial value for a measurement of flow behavior while the subject is in ventricular fibrillation by performing the sub-steps of:
(i) determining a total Doppler power for each of a plurality of frequency slices as a function of time, wherein said total Doppler power is calculated from an ultrasound signal backscattered from the fluid within the body of the subject;
(ii) determining power spectra from the determined total Doppler power whereby each of the plural frequency slices has a power spectrum over the frequencies within that frequency slice;
(iii) calculating a value for the measurement of flow behavior for each frequency slice; and
(iv) selecting at least one value from the plural calculated values as the at least one initial value;
obtaining at least one later value for the measurement of flow behavior after the subject has been defibrillated by performing sub-steps (i)-(iii) and:
(v) selecting at least one value from the plural calculated values as the at least one later value;
normalizing said at least one later value with said at least one initial value in order to obtain at least one flow index; and
determining whether there is a flow of the fluid within the body of the subject by comparing each of the at least one flow index to a predetermined threshold value, wherein there is a flow if any of the at least one flow index exceeds the predetermined threshold value.

26. The method of claim 25, wherein the ventricular fibrillation occurred any time from a fraction of a second to a few days earlier.

27. The method of claim 25, wherein said method steps are performed in a defibrillator.

28. The method of claim 27, wherein said defibrillator comprises an Automated or Semi-Automated External Defibrillator (AED).

29. A system for detecting and/or measuring, using an ultrasound device, a flow behavior of a fluid within a subject, comprising:
a processing means operative for:
determining a total Doppler power for each of a plurality of frequency slices as a function of time, wherein said total Doppler power is calculated from an ultrasound signal backscattered from the fluid within the subject;
determining power spectra from the determined total Doppler power whereby each of the plural frequency slices has a power spectrum over the frequencies within that frequency slice; and
calculating an indicia of flow behavior of the fluid within the subject for each frequency slice;
whereby the flow behavior is measured and/or detected using at least one of the calculated indicia of flow behavior of each frequency slice.

30. The system of claim 29, wherein flow behavior comprises at least one of blood perfusion, the pulse state, a heart beat rate, and/or flow and/or pulsatile activity of a colloidal or emulsion solution.

31. The system of claim 29, wherein the power spectra are determined from the total Doppler power using at least one of spectral analysis, Fourier analysis, correlation analysis, an averaged periodogram estimate, parametric methods, and/or auto-correlation analysis of the Doppler signal.

32. The system of claim 29, wherein said processing means comprises at least one of hardware, software, and firmware.

33. The system of claim 29, further comprising:
at least one ultrasonic transducer adapted to an application pad; and
a generator for exciting the at least one ultrasonic transducer.

34. The system of claim 33, wherein the generator operates in a continuous mode and/or pulsed mode.

35. The system of claim 29, further comprising:
a defibrillating unit having a controlled high voltage source; and
a controller of the defibrillating unit.

36. The system of claim 29, further comprising at least one of an electrocardiograph and a blood pressure monitor.

37. The system of claim 36, wherein the processing means cross-correlates the determined power spectra with the data collected by the at least one of an electrocardiograph and automatic blood pressure monitor in order to calculate the indicia of flow behavior.

38. The system of claim 29, wherein said system comprises a defibrillator.

39. The system of claim 38, wherein said defibrillator comprises an Automated or Semi-Automated External Defibrillator (AED).
